(19) United States
(12) Patent Application Publication
(10) Pub. No.: US 2007/0203547 A1
(43) Pub. Date: Aug. 30, 2007

(54) MEDICAL DEVICE IDENTIFICATION

(76) Inventors: Benedict James Costello, Berkeley, CA (US); Lawrence Arne, Redwood City, CA (US)

Correspondence Address: BOZICEVIC, FIELD & FRANCIS LLP (PRTS);(PROTEUS BIOMEDICAL,INC), 1900 UNIVERSITY AVENUE, SUITE 200, EAST PALO ALTO, CA, 94303, US

(21) Appl. No.: 11/611,685

(22) Filed: Dec. 15, 2006

Related U.S. Application Data
(60) Provisional application No. 60/750,983, filed on Dec. 15, 2005.

(51) Int. Cl. A61N 1/00

(52) U.S. Cl. 607/59

(57) ABSTRACT

Techniques are provided for accessing information about a medical device or a related component. One or more interrogatable identification elements are located in a medical device. An interrogation device can communicate with the identification element(s), which element(s) can store information about the medical device, such as a manufacturer name, a date of manufacture, an expiration date, configuration data, calibration data, and a list of enabled functions. In certain embodiments, the interrogation device can also store information in the identification element at any time, such as usage time or usage frequency.

### CROSS-REFERENCE TO RELATED APPLICATIONS

This application claims priority to: U.S. Provisional Application Ser. No. 60/750,983 filed on Dec. 15, 2005; the disclosure of which priority application is herein incorporated by reference.

### INTRODUCTION

### Background

Existing medical devices or components of medical devices typically have serial numbers or bar codes on them. For example, pacemaker can 101 shown in FIG. 1 is an implantable medical device that is connected to a pacing lead 102. Pacemaker can 101 has a serial number 103 etched into its surface. Lead 102 also has a serial number 104 printed on a portion of the lead, typically next to a connector 105 that connects lead 102 to can 101.

A physician may want to know the manufacturer name, the lot number, or the date of manufacture of pacemaker can 101 or lead 102. However, physicians often do not have easy access to a service manual that describes the particular functionality of pacemaker can 101 and lead 102. Information about a pacemaker can be accessed online from a database by entering serial numbers 103 and 104 located on the pacemaker can and lead.

A user may have to manually locate the serial numbers, read the serial numbers, and then enter the serial numbers into a database (e.g., using a keyboard). Because these steps are performed manually, they require time and are prone to error.

Some catheter devices have a small memory device that is used to store configuration or calibration data. The catheter's electrical controller reads the data in the memory device and configures itself so that it can use the catheter device correctly. However, these catheters have one or more extra pins that allow the memory device to communicate with the catheter's electrical controller. This solution is not ideal for implantable pacemaker leads, because these leads use standardized connectors, such as IS-1 or IS-4. Therefore, adding one or more pins is not possible. All of the available pins have dedicated functions. Space is also at an extreme premium, strongly discouraging the addition of pins.

Therefore, it would be desirable to provide a system that allows a physician to access information about a medical device, such as a pacemaker, without having to perform manual steps and without requiring an extra pin.

### Relevant Literature

U.S. Pat. Nos. 5,058,588; 5,300,120; 5,425,375; 5,674,288; 5,855,609; 5,987,343; 6,044,283; 6,377,829; 6,405,087; 6,463,310; 6,466,808; 6,600,940; and International application publication number WO06035351.

### SUMMARY

The present invention provides devices and methods for automatically accessing information about a medical device or a related component. One or more interrogatable identification elements, e.g., in the form of digital chips, that have a unique identifier are located in a medical device. An interrogation device can communicate with the identification element in a number of ways, e.g., wirelessly, using one or more existing pin connections, etc. The chip can store unique identifying information about the medical device, such as a manufacturer name, a date of manufacture, an expiration date, configuration data, calibration data, and a list of enabled functions. The interrogation device can also store information in the chip at any time, such as usage time or usage frequency.

Other objects, features, and advantages of the present invention will become apparent upon consideration of the following detailed description and the accompanying drawings, in which like reference designations represent like features throughout the figures.

### BRIEF DESCRIPTION OF THE FIGURES

FIG. 1 illustrates a pacemaker can and an implantable lead that have serial numbers printed or etched onto their surfaces, according to a prior art technique.

FIG. 2 illustrates a pacemaker can and an implantable lead that has a chip embedded in the lead body, according to an embodiment of the present invention.

FIG. 3 illustrates a schematic diagram of a controller and an implantable lead that has a digital system, according to an embodiment of the present invention.

FIG. 4 illustrates a schematic diagram of a controller and an implantable lead that has a digital system connected to wires in the lead through capacitors, according to another embodiment of the present invention.

FIG. 5 illustrates a schematic diagram of a controller and an implantable lead having a digital system that is inductively coupled to wires in the lead, according to yet another embodiment of the present invention.

FIG. 6 illustrates a schematic diagram of a controller and an implantable lead having a digital system that is coupled to wires in the lead through a transformer, according to yet another embodiment of the present invention.

FIG. 7 illustrates a schematic diagram of a controller and an implantable lead having a digital system that communicates with the controller wirelessly using antennas, according to yet another embodiment of the present invention.

FIG. 8 illustrates a schematic diagram of a controller and an implantable lead having a digital system that communicates with the controller wirelessly using oscillating magnetic fields generated by coil antennas, according to yet another embodiment of the present invention.

FIG. 9 illustrates a block diagram of a digital system that can be used with a medical device, according to another embodiment of the present invention.

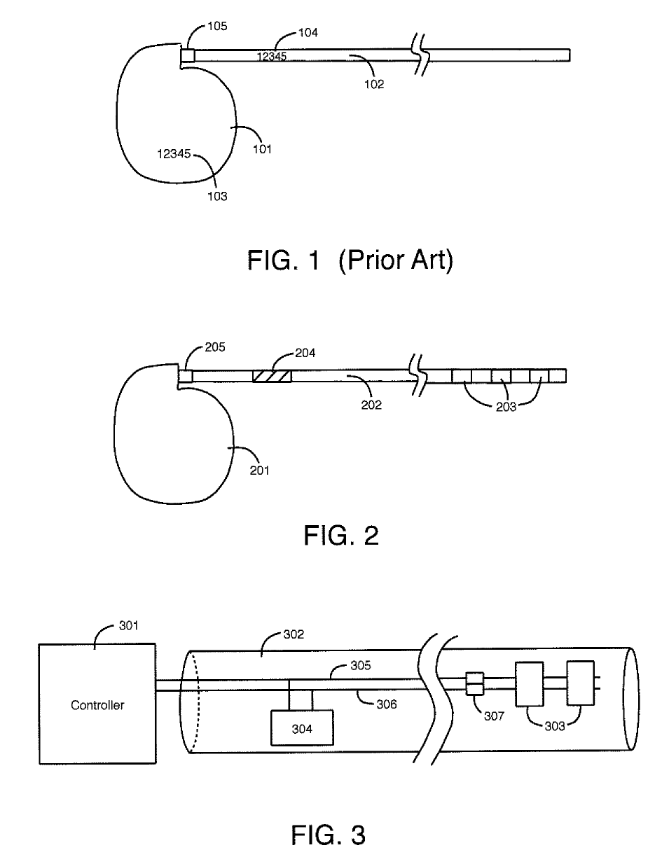

### DETAILED DESCRIPTION

According to the present invention, at least one interrogatable identification element (e.g., chip comprising an integrated circuit) is provided in a medical device. An interrogation device (e.g., a controller) can extract information about the medical device from the identification element, e.g., wirelessly, via an existing pin connection, etc. In further describing various embodiments of the invention, embodiments of the identification element of the devices will be reviewed first in greater detail, followed by a review of other aspects of the invention, including devices and kits.

Interrogatable Identification Element

An aspect of the invention is an interrogatable identification element. As the identification element is interrogatable, it can be interrogated or queried by an interrogation device, e.g., which interrogation device may be in vivo or ex vivo and may communicate with the element wirelessly or via one or more wires. As such, the interrogation device can communicate with the identification element to obtain information from the element, which information may include unique identifier information.

The identification element can store a variety of machine-readable information about the medical device with which it is associated. For example, a manufacturer name, a date of manufacture, a lot number, a serial number, an expiration date, and a list of enabled functions can be stored in memory on an identification element associated with a medical device. The interrogation device can read the data from the memory and display the data to a user, e.g., physician or other health care professional, upon request. This technique eliminates the chances of human error interfering with a proper identification of the medical device and saves time.

According to some embodiments of the present invention, data can be stored in memory in a chip that is part of a medical device at any time. For example, a controller in an external device can keep track of the number of times that the medical device has been used. The controller can also keep track of the amount of time that the medical device has been used. The controller can store this information in the chip from time-to-time, and can use the stored information to determine when the medical device has exceeded its useful lifespan. After a maximum usage frequency or usage time has been exceeded, the controller or other device can disable the medical device or warn a user against using it.

FIG. 9 illustrates a simplified block diagram of a digital system 901 in a medical device, according to an embodiment of the present invention. Digital system 901 is an example of digital systems 304, 402, 502, 602, 704, and 804, where these elements are reviewed in greater detail below. Digital system 901 is merely one example of a digital system that can be used in a medical device and is not intended to limit the scope of the present invention.

Digital system 901 includes a physical layer interface 902, an encoding/decoding block 903, a controller block 904, and a memory block 905. Physical layer interface 902 contains the circuitry necessary to drive the signaling onto a transport mechanism used in a particular implementation, such as the wired, wireless, direct, inductive, and capacitive coupling embodiments described above.

Encoding/decoding block 903 encodes and decodes data for transmission to and from an external interrogation device. Block 903 can implement error detection and correction algorithms on data transmitted to and from system 901 using well-known error correction techniques. Block 903 can also implement data compression and decompression on the data to reduce its size during transmission. Block 903 can also encrypt and decrypt the transmitted data to provide data security. Block 903 can also perform data integrity encryption to control who can write and re-write data into memory 905. Block 903 can use a variety of encoding/decoding schemes, such as fsk, ask, psk, nze, Manchester, etc.

Controller block 904 extracts commands and data from a bit stream received from the external interrogation device (e.g., controllers 301, 401, 501, 601, 701, or 801). Controller 904 can act upon the received commands to perform a variety of functions. For example, controller block 904 can read data from and write data to non-volatile and volatile memory in block 905. Memory block 905 can be built around one or more memory technologies to suit the particular application. For example, memory block 905 can include any one or more of the following technologies, ROM, PROM, EPROM, EEPROM, FLASH, FEPROM, DRAM, and SRAM. Data stored in memory 905 can be secured through encryption or password protection.

An identification element chip that is part of an implantable medical device may be protected from the environment inside a patient to prevent damage to the chip. For example, the chip or the body of the implantable medical device can be encased in a material that provides an effective barrier to moisture and saline. In certain embodiments, the chip may be “hermetically sealed,” e.g., as described in PCT application serial PCT/US2005/046815 titled “Implantable Hermetically Sealed Structures” and filed on Dec. 22, 2005; and provisional application Ser. No. 60/791,244 filed Apr. 12, 2006; the description of hermetically sealed structures provided in these applications being specifically incorporated herein by reference.

The identification elements of the present invention may be used with any of a variety of different types of medical devices, where the medical devices are, in certain embodiments, implantable medical devices. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human. The function of the implantable medical devices of the invention may vary widely, including but not limited to: cardiac devices, drug delivery devices, analyte detection devices, nerve stimulation devices, etc. Illustrative embodiments of various types of implantable medical devices of the invention are reviewed in greater detail below. According to certain embodiments of the present invention, a chip or a digital system including a chip can be used with other types of medical devices. Examples of medical devices that can employ techniques of the present invention include cochlear implant devices, retinal implant devices, diaphragm pacemakers, implantable EKG devices, implantable glucose sensors, physiological sensors, e.g., physiological pressure sensors, any type of medical device having a can and implantable leads, and other types of medical sensors or regulating devices. As another example, the techniques of the present invention can be applied to implantable stimulation devices, e.g., gastro-stimulation devices and neuro-stimulation devices.

In certain embodiments, the implantable medical device is a cardiovascular device. By cardiovascular device is meant a device that is employed in the treatment of, e.g., in the delivery of therapeutic stimulation, in the sensing of hemodynamic parameters, etc., some aspect of a cardiovascular disease. In cardiovascular device embodiments, the device or at least some portion thereof may be configured to be positioned in a cardiovascular structure, e.g., in or on the heart, in a vessel, such as an artery or vein, etc.

In certain embodiments as developed more fully below in connection with the review of the figures, the cardiovascular device is a lead, e.g., a cardiovascular lead, which lead includes at least one of the identification elements of the invention and at least one effector.

The medical devices may include a variety of different effector elements. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled; “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled; “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Vascular Leads

Embodiments of the invention also include medical carriers that include one or more electrode satellite structures, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., a standardized connector, such as an IS-1 or IS-4 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, etc.

In certain embodiments of the subject systems, one or more sets of effectors, e.g., electrodes, satellites are coupled, e.g., electrically coupled, to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or fewer wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference.

In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

In certain embodiments, the effector is an electrode structure. Electrode effectors that are present on the lead may vary, and may include a single electrode or two or more electrodes, e.g., present as a segmented electrode structure. By segmented electrode structure is meant an electrode structure that includes two or more, e.g., three or more, including four or more, disparate electrode elements. Embodiments of segmented electrode structures are disclosed in application Ser. No. PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various segmented electrode structures of these applications being herein incorporated by reference. In these embodiments, the support may include a recess for each electrode element of the segmented electrode structure. As such, the support may include 2 or more, 3 or more, 4 or more, etc., where each recess is configured to receive an electrode element (i.e., an electrode inset).

In certain embodiments, the electrode structures are “addressable” electrode structures. Addressable electrode structures include structures having one or more electrode elements directly coupled to control circuitry, e.g., present on an integrated circuit (IC). Addressable electrode structures include satellite structures that include one more electrode elements directly coupled to an IC and configured to be placed along a lead. Examples of addressable electrode structures that include an IC are disclosed in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various addressable electrode structures of these applications being herein incorporated by reference.

FIG. 2 illustrates an implantable pulse generator, e.g., in the form of a pacemaker, system according to a first embodiment of the present invention. The pacemaker of FIG. 2 includes a pacemaker can 201 that is connected to an implantable multi-electrode pacing lead 202 through a connector 205. Lead 202 typically has a small diameter. Examples of possible lead diameters are in the ranges of about 0.5-5 mm, about 0.75-3 mm, or more preferably between about 1 mm-2 mm. Lead 202 includes a chip 204 and three electrodes 203. Electrodes 203 are multiplexed so that pacemaker can 201 can send stimuli to any or all of the electrodes under computer control. Pacemaker can 201 can include multiple leads. However, only one lead is shown in FIG. 2 for simplicity. Each of the leads can include a separate chip, if desired.

Can 201 needs to have details about lead 202, such as how many electrodes it has and the addresses of the electrodes. However, can 201 does not necessarily have all of the information it needs about lead 202 programmed into its memory, because can 201 is designed to operate with different types of leads that may have a different number of electrodes.

Identification element in the form of chip 204 can store information about pacing lead 202 that can be read from an external interrogation device. For example, chip 204 can store a serial number, a lot number, a manufacturer name, a date of manufacture, the number of electrodes on lead 202, an expiration date, and compatibility information. The information stored on the chip is, in certain embodiments, unique to the device with which the chip is associated, where by unique is meant that the information pertains to that device alone, and not to any other device, and is therefore analogous to a “fingerprint” for that device. When addressable components are part of a device's construction, Chip 204 may store the ID numbers so they may be addressed appropriately. Chip 204 can also store other information, such as the impedance of each electrode. The impedance of each electrode can be used to self-diagnose a failure or a change in the condition of the lead.

An external interrogation device, such as a controller, can communicate with chip 204. The external interrogation device can read the information stored in memory in chip 204. The external interrogation device can be located, e.g., in can 201, in a programmer that programs can 201, or in a separate housing, and therefore may be in vivo or ex vivo.

The external interrogation device can also store information in memory in chip 204 at any time, such as the usage time and the usage frequency of lead 202. There is an ongoing tendency among medical practitioners to re-use single or limited-use medical devices beyond the lifespan intended by the manufacturer in an attempt to save money. This practice can endanger patients and reduces the manufacturer's revenue.

To address this problem, an external interrogation device can track the usage of lead 202 and store the data in memory within chip 204, as described above. The memory can store the duration of usage of lead 202 and/or a usage count (i.e., the number of times lead 202 has been used). This information can be used to prevent the re-use of single usage devices. This information can also be used to prevent the re-use of devices that have exceeded their maximum safe usage duration or count.

When lead 202 is connected to can 201, an external interrogation device interrogates chip 204 for its usage history. If the usage history stored in chip 204 permits additional use, installation continues, and can 201 updates the usage history stored in chip 204. If the stored usage history indicates that the lead has exceeded the maximum number of permitted uses or the maximum duration of use, the re-use of the lead is blocked.

Chip 204 can also store software code or algorithms. For example, a new lead may contain functionality that is not envisioned when a pacemaker can or other external medical device is originally manufactured. Memory in chip 204 can store a code patch that allows a microprocessor or micro-controller in an external medical device to implement the new functionality As another example, chip 204 can store algorithms that allow an external medical device to process or interpret data from satellite devices (e.g., sensors or electrodes) on lead 202. At the time of connection, the code is downloaded from chip 204 to the external medical device, where is it loaded into program storage.

Chip 204 can also store configuration data that can be read and used by an external medical device, such as pacemaker can 201. The configuration data can indicate what type and how many satellite devices (e.g., sensors or electrodes) are on lead 202. For example, the configuration data can indicate that there are 3 electrodes on lead 202. As another example, the configuration data can indicate that the lead has two pressure sensors. The configuration data stored in chip 204 can also include the address of each satellite device on the lead. The configuration data stored in chip 204 allows an external controller to determine what functions lead 202 can perform without requiring human input or intervention.

Chip 204 can also store calibration data that allows an external medical device, such as a pacemaker can, to make use of the satellite devices on the lead. As an example, the calibration data can indicate the impedance of electrodes or the impedance of sensors on the lead. As yet another example, if the lead has a pressure sensor, chip 204 can store calibration data that can be used to convert voltages from the pressure sensor into pressure values.

One specific example of chip 204 is a radio frequency identification chip (RFID). RFID chips typically contain memory and unique serial numbers. An RFID chip in lead 202 can communicate wirelessly with an interrogation device using RF signals. Some types of RFID chips are ideal for use in an implantable device such as a pacemaker lead, particularly if they are small in size, have non-volatile memory, and harvest their energy from the interrogation signal. Also, some types of RFID chips can communicate with an interrogation device through a high loss wireless link. Examples of commercial RFID chips that can be used in a medical device according to the present invention are manufactured by Texas Instruments Inc. and Atmel Corp.

Chip 204 such as an RFID chip or any other type of memory chip can contain any desirable amount of memory. For example, the memory storage capacity of chip 204 can be in the range of about 0.25 kilobyte to 100 megabytes, such as in the range of about 1-10 kilobytes, about 3-8 kilobytes, or about 7 kilobytes.

FIG. 3 illustrates a schematic diagram of a portion of a medical device with an implantable lead that has a digital system 304, according to an embodiment of the present invention. Controller 301 is part of an external portion of the medical device. For example, controller 301 can be part of a pacemaker can or part of a programmer device that is used to program the pacemaker can.

The medical device also includes a lead 302 that has a flexible housing. Controller 301 is coupled (directly or indirectly) to lead 302 through two wires 305 and 306. Wires 305 and 306 are embedded within the body of lead 302 as shown in FIG. 3. Lead 302 has two effectors (also referred to herein as “endo-factors”) 303 near its distal end. Controller 301 is also coupled to endo-factors 303 through wires 305 and 306. Endo-factors 303 can be, for example, electrodes for stimulating tissue or sensing electrical fields, or sensors for measuring parameters such as temperature, pressure, blood flow, or other physiological parameters. If controller 301 is part of a programmer device, controller 301 is not directly connected to wires 305 and 306 as shown in FIG. 3. Instead, controller 301 communicates indirectly with devices in the lead through a wireless link with a pacemaker can that is connected to the lead.

Controller 301 is coupled to an identification element, e.g., the form of a digital system 304, through wires 305 and 306 in FIG. 3. Digital system 304 can be embedded in any portion of lead 301, e.g., nears its proximal or distal ends or in the middle of lead 301. Alternatively, digital system 304 can be located in a connector (not shown) between lead 301 and an external device (e.g., an IS-1 connector), in a separately packaged device in the lead, or in another device on the lead. Digital system 304 includes one or more digital chips. For example, digital system 304 can include a general-purpose processor, a signal processor, or a controller that receives and responds to instructions from controller 301.

Controller 301 can send electrical signals along wires 305 and 306 to communicate with digital system 304. Digital system 304 can transmit data to controller 301, e.g., in response to communications from controller 301. Thus, controller 301 can communicate with digital system 304 without using any extra pin connections in the connector (e.g., connector 205). Signaling between controller 301 and the digital system 304 may use various encoding schemes, e.g., FSK, ASK, etc.

Digital system 304 can transmit requested information to controller 301, such as configuration data and/or calibration data. Digital system 304 can also store and transmit manufacturing parameters to controller 301, such as a model number of the lead, a batch number, a date of manufacture, a manufacturer name, etc. Controller 301 can display the data received from digital system 304 to a user on a display screen.

Digital system 304 can also include memory such as a ROM, PROM, EPROM, EEPROM, FLASH, FEPROM, DRAM, SRAM, etc. Digital system 304 preferably includes at least one block of non-volatile memory such as ROM, PROM, EPROM, EEPROM, or FLASH. However, digital system 304 may also include volatile memory such as SRAM or DRAM. The memory in digital system 304 can be part of a separate chip or part of an integrated circuit that includes a controller or other circuitry.

In certain embodiments, controller 301 can communicate with digital system 304 without stimulating or turning on endo-factors 303. For example, controller 301 can communicate with digital system 304 using a low voltage that does not stimulate endo-factors 303 or that does not cause endo-factors 303 to send a charge to the tissue (e.g., about 0.1-1.0 volts). As another example, controller 301 can communicate with digital system 304 using short duration pulses (e.g., less than about 1000 microseconds, less than about 100 microseconds, less than about 10 microseconds, less than 1 about microsecond, or less than about 0.1 microsecond). Alternatively, lead 302 can include switches 307 on wires 305 and 306 that can couple and decouple endo-factors 303 from controller 301. Controller 301 turns off switches 307 when communicating with digital system 304 to prevent the communications signals from turning on endo-actors 303 or other sensors on lead 302. Switches 307 can be, for example, pass transistors.

Where endo-factors (203 or 303) or sensors have a digital interface/signaling between them and the controller, that interface may be used to address the digital system with each device type having a unique ID. Each device type uses that ID to recognize which communications packets are intended for it. Alternatively, different device types may use different communications schemes and rely upon a combination of addressing and error detection/correction codes (parity bits, CRCs, etc.) to identify which signaling is intended for it.

According to some embodiments of the present invention, the wire(s) of an implantable medical device can be indirectly coupled to a chip or digital system using capacitive or inductive coupling.

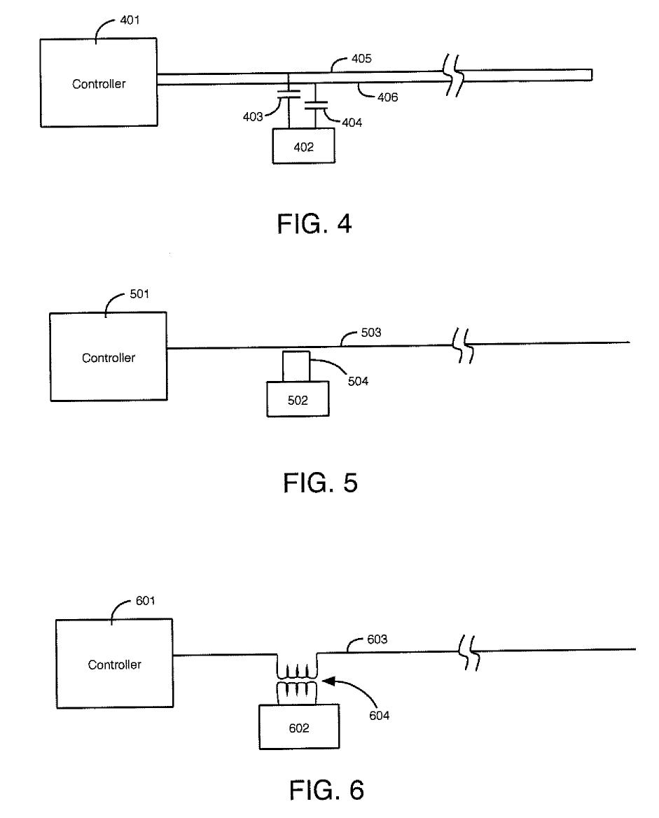

FIG. 4 illustrates a schematic diagram of a portion of a medical device with an implantable lead that has a digital system 402, according to another embodiment of the present invention. The implantable medical device includes a controller 401 that is part of an external portion of the device. For example, a controller 401 can be part of a pacemaker can or a programmer that is used to program the pacemaker can.

The implantable medical device also includes a lead having a flexible body. The body of the lead is not shown in FIG. 4. The lead includes two wires 405 and 406 that couple controller 401 to electronic components in the lead. Specifically, wires 405 and 406 couple controller 401 to a digital system 402. Digital system 402 can be, for example, embedded within the body of the lead, in a connector that connects the lead with an external device, in a separately packaged device in the lead, or in another device on the lead.

In the embodiment of FIG. 4, digital system 402 is coupled to wires 405 and 406 through capacitors 403 and 404. It may be beneficial to indirectly connect digital system 402 to wires 405 and 406 to improve electrical reliability and to increase patient safety. Digital system 402 includes one or more chips such as memory a controller, etc., as described above with respect to the previous embodiments. As with the previous embodiment, controller 401 can communicate with digital system 402 without the need for an extra pin.

FIG. 5 illustrates a schematic diagram of a portion of a medical device with an implantable lead that has a digital system 502, according to another embodiment of the present invention. The medical device includes an external controller 501 and a lead that has a single wire 503 and a digital system 502. Digital system 502 can be, for example, embedded in the body of the lead, in a connector, in a separately packaged device in the lead, or in another device on the lead. The body of the lead is not shown in FIG. 5.

Wire 503 is inductively coupled to digital system 502 through an inductive coil 504. In the embodiment of FIG. 5, the simplest implementation of a coil is just two wires running parallel to each other. These two wires simulate a transformer that can couple electrical energy between wire 503 and digital system 502. The inductive connection between wire 503 and digital system 502 can also provide the benefits of an indirect connection that are mentioned above.

FIG. 6 illustrates a schematic diagram of a portion of a medical device with an implantable lead that has a digital system 602, according to yet another embodiment of the present invention. The body of the lead is not shown in FIG. 6. The medical device includes an external controller 601, as well as a lead that has a single wire 603 and a digital system 602. Wire 603 is inductively coupled to digital system 602 through a transformer 604.

According to further embodiments of the present invention, the wire(s) of a medical device can be decoupled from the chip using a pair of antennas. In these embodiments, the requests sent by an external controller and the replies from the chip can be transmitted wirelessly without any electrical contact between the chip and the controller. These embodiments improve patient safety and electrical reliability by eliminating the need for a controller to apply a voltage or current through a lead to communicate with the chip.

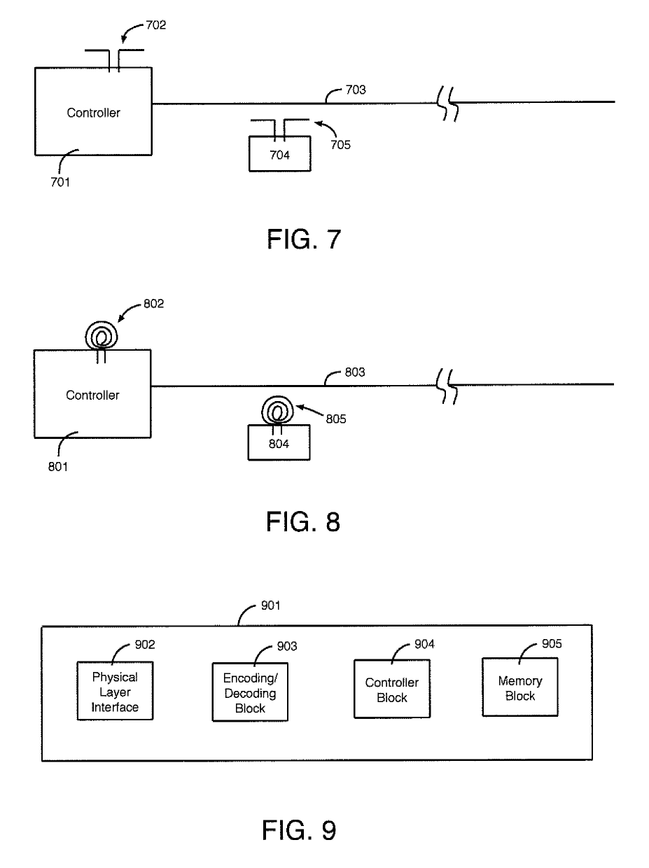

FIG. 7 illustrates a schematic diagram of a portion of a medical device with an implantable lead that has a digital system 704, according to another embodiment of the present invention. Digital system 704 can be, for example, embedded in the body of the lead (not shown), in a connector, in a separately packaged device in the lead, or in another device on the lead. An external controller 701 communicates with digital system 704 through a pair of antenna 702 and 705. Controller 701 can wirelessly transmit signals to and receive signals from digital system 704 using antenna 702. Digital system 704 can wirelessly transmit signals to and receive signals from controller 701 using antenna 705. Thus, controller 701 can communicate with digital system 704 without sending signals on lead wire 703. Thus, there is no need for an extra pin in the connector to communicate with digital system 704.

In FIG. 7, the antenna 702 and 705 are shown as dipole antennas for illustrative purposes. However, any type of antennas can be used to allow communication between digital system 704 and controller 701. For example, antennas 702 and 705 can have dimensions in the range of about 10μ to 10 cm, about 100μ to 2 mm, and about 1 mm. The antennas can transmit signals in the range of about 1 MHz-300 GHz, about 10 Mhz-100 GHz, about 1-20 GHz, more specifically about 2.4 GHz. Controller 701 can communicate through antenna 702 using, for example, radio waves or oscillating electrical fields.

FIG. 8 illustrates a schematic diagram of a portion of a medical device with an implantable lead that has a digital system 804, according to yet another embodiment of the present invention. Digital system 804 can be, for example, embedded in the body of the lead (not shown), in a connector, in a separately packaged device in the lead, or in another device on the lead.

An external controller 801 communicates with digital system 804 through a pair of antennas 802 and 805. Controller 801 can wirelessly transmit signals to and receive signals from digital system 804 using antenna 802. Digital system 804 can wirelessly transmit signals to and receive signals from controller 801 using antenna 805. In the embodiment of FIG. 8, antennas 802 and 805 are coil antennas that communicate via oscillating magnetic fields. Thus, controller 801 can communicate with digital system 804 without sending signals on lead wire 803 or using an extra pin. The body of the lead is not shown in FIG. 8.

Implantable Pulse Generators

Embodiments of the invention further include implantable pulse generators, such as the pacemakers described above and depicted in FIGS. 2 to 8. Implantable pulse generators may include: a housing which includes a power source and an electrical stimulus control element; one or more vascular leads as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

Summarizing aspects of the above description, in using the implantable pulse generators of the invention, such methods include implanting an implantable pulse generator e.g., as described above, into a subject; and using the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in application Ser. No. PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various methods of operation of these applications being herein incorporated by reference and applicable for use of the present devices.

Systems

Also provided are systems that include implantable medical devices of the invention, e.g., as described above. The systems may include an implantable medical device as described above, and an interrogation device, where the identification element of the implantable medical device communicates with the interrogation device, e.g., through a wire based, e.g., pin, communication protocol or a wireless communication protocol.

The systems of the invention may be viewed as systems for communicating information within the body of subject, e.g., human, where the systems include both a first implantable medical device, such as lead device described above, that includes a transceiver configured to transmit and/or receive a signal, including a signal containing data from the identification element of the device; and a second device comprising a transceiver configured to transmit and/or receive a signal, e.g., an interrogation device. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may be transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Methods

Also provided are methods of using devices and systems of the invention, e.g., as described above. In certain embodiments, the methods are methods of identifying an implantable medical device. In such embodiments, the methods include interrogating, e.g., by communicating a query signal to, an implantable medical device that includes an interrogatable identification element with a unique identifier to obtain said unique identifier, e.g., in the form of a communicated response from the identification element; and using the obtained unique identifier to identify said medical device. In certain embodiments, the methods are methods of automatically accessing information about the implantable medical device. As reviewed above, the interrogation step can be done from an external interrogation device or an internal interrogation device, and can be performed using any convenient form of communication, including wire based and wireless communication.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Kits

As summarized above, also provided are kits that include various components, e.g., as described above. The kits include at least a medical device with a unique identifier that can be interrogated, e.g., as described above. In certain embodiments, the kits may also include an interrogation device, e.g., as described above. The kits and systems may also include a number of optional components that find use with the medical device identification system, including but not limited to, delivery devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, component containers and the like. In the subject kits, the one or more components are present in the same or different containers, as may be convenient or desirable.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events,

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. An implantable medical device comprising an interrogatable identification element with a unique identifier and at least one effector.

2. The implantable medical device according to claim 1, wherein said interrogatable identification element comprises a memory, a controller, and an encoding/decoding block.

3. The implantable medical device according to claim 2, wherein said memory stores unique device identification data.

4. The implantable medical device according to claim 3, wherein said memory stores unique device usage data.

5. The implantable medical device according to claim 2, wherein said interrogatable identification element is configured to communicate with an interrogation device.

6. The implantable medical device according to claim 5, wherein said interrogatable identification element is configured to communicate wirelessly with said interrogation device.

7. The implantable medical device according to claim 5, wherein said interrogatable identification is configured to communicate with said interrogation device using at least one wire.

8. The implantable medical device according to claim 5, wherein said interrogation device is an ex vivo device.

9. The implantable medical device according to claim 5, wherein said interrogation device is an implantable device.

10. The implantable medical device according to claim 1, wherein said device includes at least at least two effectors.

11. The implantable medical device according to claim 10, wherein said effector comprises an electrode.

12. The implantable medical device according to claim 1, wherein said implantable medical device is a device selected from the group consisting of a cochlear implant device, retinal implant device, diaphragm pacemaker, implantable glucose sensor, physiological sensor and cardiovascular device.

13. The implantable medical device according to claim 12, wherein said physiological sensor is a physiological pressure sensor.

14. The implantable medical device according to claim 12, wherein said implantable medical device is a cardiovascular device.

15. The implantable medical device according to claim 14, wherein said cardiovascular device is a lead comprising at least two effector satellites.

16. The implantable medical device according to claim 15, wherein said lead includes a single pin connector at its proximal end.

17. The implantable medical device according to claim 16, wherein said connector is an IS-1 connector.

18. A medical system comprising:
an interrogation device; and
an implantable medical device comprising an interrogatable identification element with a unique identifier.

19. The system according to claim 18, wherein said interrogatable identification element comprises a memory, a controller, and an encoding/decoding block.

20. The system according to claim 19, wherein said memory stores unique device identification data.

21. The system according to claim 20, wherein said system is a cardiovascular system.

22. The system according to claim 21, wherein said implantable medical device is present on a cardiovascular lead.

23. The implantable medical device according to claim 22, wherein said implantable medical device comprises said interrogatable identification element and at least one effector present on said cardiovascular lead.

24. The implantable medical device according to claim 23, wherein said cardiovascular lead comprises at least two effector satellites.

25. The implantable medical device according to claim 24, wherein said lead includes a pin connector at its proximal end.

26. The implantable medical device according to claim 25, wherein said connector is a IS-1 connector.

27. The implantable medical device according to claim 22, wherein said interrogation device comprises an implantable pulse generator.

28. A method for identifying an implantable medical device, said method comprising:
interrogating an implantable medical device comprising an interrogatable identification element with a unique identifier to obtain said unique identifier; and
using said obtained unique identifier to identify said medical device.

29. The method according to claim 28, wherein said implantable medical device is implanted in a subject.

30. The method according to claim 29, wherein said interrogating comprises employing an ex vivo interrogation device.

31. The method according to claim 29, wherein said interrogating comprises employing an in vivo interrogation device.

32. A kit comprising:
an implantable medical device comprising an interrogatable identification element with a unique identifier; and
instructions for using said device.

33. The kit according to claim 32, wherein said kit further comprises an interrogation device.
